(19) United States
(12) Patent Application Publication
(10) Pub. No.: US 2022/0315992 A1
(43) Pub. Date: Oct. 6, 2022

(54) METHOD FOR IDENTIFICATION OF URINE DRUG TESTING SAMPLE

(71) Applicant: ID Match, LLC, Denver, CO (US)

(72) Inventors: Lynn PUANA, Denver, CO (US); Cayce AKSOY, Kailua-Kona, HI (US)

(73) Assignee: ID Match, LLC, Denver, CO (US)

(21) Appl. No.: 17/512,713

(22) Filed: Oct. 28, 2021

Related U.S. Application Data
(60) Provisional application No. 63/171,257, filed on Apr. 6, 2021.

(51) Int. Cl. C12Q 1/6809; C12Q 1/682; C12Q 1/686; C12Q 1/6806

(52) U.S. Cl. CPC C12Q 1/6809; C12Q 1/682; C12Q 1/686; C12Q 1/6806; H01J 49/164

(57) ABSTRACT

A method for identification of a urine sample and for verification of a donor identity of a urine drug testing sample to rule out adulteration or substitution in urine drug testing, comprising: preparing a urine drug testing sample and a reference sample with a confirmed donor identity, extracting or releasing DNA from the urine drug testing sample and the reference sample, amplifying and genotyping the extracted or released DNA from both samples; analyzing and comparing the DNA from the urine drug testing sample to the DNA from the reference sample; and based upon the analysis and comparison results, determining and reporting a match or mismatch between a donor identity of the urine drug testing sample and the donor identity of the reference sample. The donor identity of the reference sample is confirmed to be a person providing the urine drug testing sample.

### CROSS-REFERENCE TO RELATED APPLICATIONS

The present patent application claims benefit and priority to U.S. Provisional Patent Application No. 63/171,257 entitled “METHOD FOR IDENTIFICATION OF URINE DRUG TESTING SPECIMEN” filed on Apr. 6, 2021 which is hereby incorporated by reference into the present disclosure.

### BACKGROUND

Drug testing is an important deterrent for drug abuse. Urine drug testing is the most widely used method for detecting substances (therapeutic and illicit) in a person's body.

Urine is the preferred medium for drug testing because results are cost effective and accurate, with longer windows of detection than saliva or blood, and faster turnaround times than hair. Because of the consequences associated with unexpected urine drug test results, individuals may go to great lengths to avoid detection.

Sample adulteration and substitution (with substances to alter the results as well as other people's urine or synthetic urine) are common but can be combatted on some level with physical observations (temperature, color, odor, etc.) and chemical tests (nitrites, oxidants, pH, specific gravity, creatinine and glutaraldehyde, etc.). However, even with observed collection of urine specimens there is still plenty of room for fake samples or not the actual person's sample to be used as to avoid being caught with drugs in their own urine specimen. This ability to properly identify the urine sample as actual human urine and as that individual's (not someone else's) allows the entity requiring the test to have 100% certainty of the results being accurate. There is not currently a cost effective, expeditious method for confirming the identity of a urine sample.

Therefore, there is a need for an economical and timely way to report with absolute confidence that a urine sample collected from a patient/client was, in fact, voided by said patient/client.

### SUMMARY

The present invention relates to a method for identification of a urine sample, for example, a method for verification of a donor identity of a urine drug testing sample to rule out adulteration or substitution in urine drug testing. The method comprises: preparing a urine drug testing sample and a reference sample with a confirmed donor identity; extracting, or releasing DNA, from the urine drug testing sample and the reference sample; amplifying and genotyping the extracted or released DNA from the both samples; after the genotyping, analyzing and comparing the DNA from the urine drug testing sample to the DNA from the reference sample; and based upon the analysis and comparison results, determining and reporting a match or mismatch between a donor identity of the urine drug testing sample and the confirmed donor identity of the reference sample. The donor identity of the reference sample is confirmed to be a person providing the urine drug testing sample.

In some embodiments, the donor identity of the reference sample can be confirmed through polymerase chain reaction (PCR) and mass spectrometry (MS).

In some embodiments, the reference sample can be a buccal cell sample, a urine sample, a whole blood tissue sample, a saliva sample and/or an oral rinse sample from the person providing the urine drug testing sample. The DNA from a urine drug testing sample can be gDNA from epithelial cells excreted into the urine or from white blood cells in the urine.

In a preferred embodiment, the reference sample can be a buccal cell swab, and the DNA from a urine drug testing sample can be gDNA from epithelial cells excreted into the urine.

In a further aspect, extracting DNA from the urine drug testing sample and the reference sample can be performed by commercially available DNA extraction kits, with or without automated DNA extraction instruments. In a preferred embodiment, commercially available DNA extraction kits which can be intended for buccal cell DNA extraction can be used in conjunction with automated DNA extraction instruments. Additionally, DNA from the urine drug testing sample and the reference sample can be extracted on the same plate at the same time with the same DNA extraction kit. Specifically, the extracting step may comprise digesting cells from both samples with Proteinase K mix, and performing DNA purification according to the User Guide of the commercially available DNA extraction kits.

In another aspect, the DNA from the urine drug testing sample and the reference sample can be released through a one-step lysis procedure to free cellular contents in the samples. In a preferred embodiment, the one-step lysis procedure comprises using a lysis buffer to break open the cells from the urine drug testing sample and the reference sample, as well as nuclei in each of the cell types, thus releasing the DNA for amplification and subsequent analysis and comparison. The lysis buffer can be formed of enzymes and detergents.

In a further aspect, the amplification of extracted DNA from both samples can be performed by polymerase chain reaction (PCR).

In a further aspect, the analysis and comparison of amplified DNA from the urine drug testing sample and the reference sample can be performed by any method for analyzing single nucleotide polymorphisms (SNPs), for example, one or more selected from the group of MS (mass spectrometry), DNA sequencing, molecular beacons, SNP microarrays, PCR-based methods, random amplified polymorphic detection (RAPD), restriction fragment length polymorphism (RFLPI), amplified fragment length polymorphism detection (AFLPD), and allele specific oligonucleotide (ASO) probes. More preferably, the analysis and comparison step are performed by matrix assisted laser desorption ionization-time of flight mass spectrometry (MALDI-TOF MS).

In summary, by matching the DNA in a urine sample to the DNA in a sample with no associated question of donor identity through PCR and mass spectrometry after a modified singular DNA sample preparatory method, the present invention provides an economical and timely method for identification of a urine drug testing sample. The presented herein approach has a great potential for providing a faster, cheaper and more accurate and efficient way to rule out adulteration or substitution in observed collection urine specimen collection. Furthermore, the methods of the invention are not only adaptable for sample identification in clinical toxicology, but also adaptable for workplace drug testing spaces.

### BRIEF DESCRIPTION OF THE DRAWINGS

The accompanying drawings illustrate various embodiments of systems, methods, and various other aspects of the embodiments. Any person with ordinary art skills will appreciate that the illustrated element boundaries (e.g., boxes, groups of boxes, or other shapes) in the FIGURES represent an example of the boundaries. It may be understood that, in some examples, one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a flow diagram of a method for verification of a donor identity of a urine drug testing sample. The donor identity of the reference sample is confirmed to be a person providing the urine drug testing sample.

### DETAILED DESCRIPTION OF THE EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

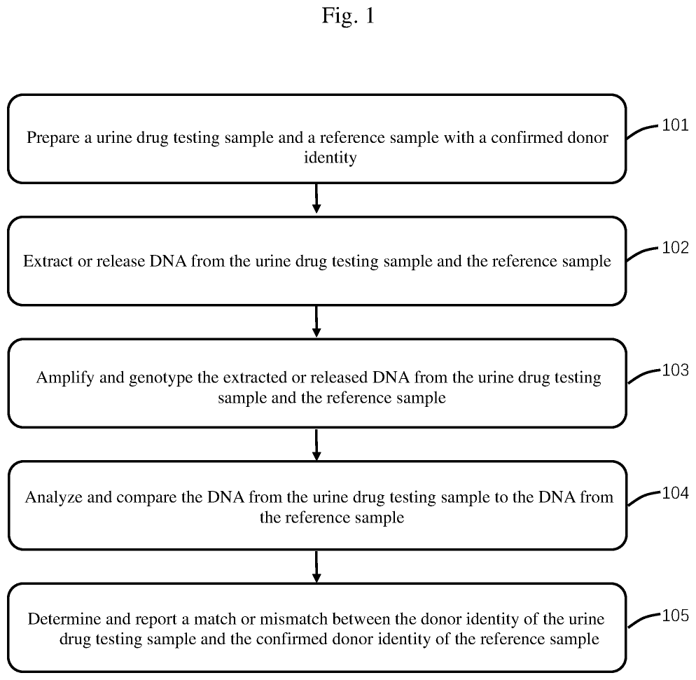

FIG. 1 illustrates a flow diagram of a method for verification of a donor identity of a urine sample. In some exemplary embodiments, the urine sample may be a urine drug testing sample, although it is envisioned that any urine sample may be utilized or tested with the embodiments. For example, the method begins with preparing a urine drug testing sample and a reference sample with a confirmed donor identity 101, the donor identity of the reference sample is confirmed to be a person providing the urine drug testing sample; then extracting or releasing DNA from the urine drug testing sample and the reference sample 102, and amplifying and genotyping the extracted or released DNA from the both samples 103; then, analyzing and comparing the DNA from the urine drug testing sample to the DNA from the reference sample 104; and based upon the analysis and comparison results, determining and reporting a match or mismatch between a donor identity of the urine drug testing sample and the confirmed donor identity of the reference sample 106. By doing so, a report with absolute confidence that the urine drug testing sample collected by a patient/client is, in fact, voided by said person may be provided.

Exemplary embodiments described herein may relate to a modified preparatory method of a reference sample with the confirmed donor identity and a urine drug testing sample for DNA extraction. Such sample with confirmed donor identity may be buccal cells on a cotton swab—the most inexpensive and easy sample to collect for sample identification.

In some embodiments, in order to extract DNA from buccal cell samples, a single kit and a modified single extraction method for extracting DNA from both sample types at the same time, on the same plate(s) is provided. In some preferable embodiments, the Applied Biosystems MagMAX™ DNA Multi-Sample Ultra 2.0 Kit from Thermo Fisher Scientific Inc.—a kit designed to be used with KingFisher Flex automation for high throughput isolation of DNA from “whole blood, saliva, buffy coat, and buccal swabs” can be purchased and used. The kit is not designed for nucleic acid extraction from urine, but the inventor found that DNA was able to be extracted from epithelial cells excreted into urine with the aforementioned kit by the following steps. First urine particulates were isolated through centrifugation (5 mL urine in a 15 mL conical test tube at 6000 rpm for 20 minutes). After centrifugation, the supernatant was discarded and the urine pellets were resuspended in 480 uL proteinase K mixture, prepared in accordance with the Thermo Fisher Scientific Inc.'s Applied Biosystems MagMAX™ DNA Multi-Sample Ultra 2.0 Kit User Guide, written and intended for buccal cell DNA extraction.

The MagMAX™ DNA Multi-Sample Ultra 2.0 Kit User Guide is described in the Thermo Fisher Scientific Inc.'s Applied Biosystems presentation entitled “MagMAX™ DNA Multi-Sample Ultra 2.0 Kit USER GUIDE” published in 2019, hereby incorporated by reference in its entirety with the following modifications.

Sufficient Proteinase K Mix was prepared according to the following table (in the order they are listed, in accordance with the User Guide: Prepare samples and digest with Proteinase K step 1.c., page 3), then the components were gently inverted to thoroughly mix.

| Component ^{[1]} | Volume per well | | Volume per plate (96 samples) | |
|---|---|---|---|---|
| Enhancer Solution | 40 | μL | 4.22 | mL |
| PBS | 400 | μL | 42.24 | mL |
| Proteinase K | 40 | μL | 4.22 | mL |
| Total volume | 480 | μL | 50.68 | mL |

^{[1]} Pipet the components in the order they are listed in the table

After pellet resuspension, the resuspended pellets were transferred into a 96 deep-well plate according to a batch plate map (one sample per well). Then the corresponding buccal swabs were equilibrated to room temperature and the swabs were placed into wells on the same 96 deep-well plate (one per well according to the same batch plate map, in accordance with the MagMAX™DNA Multi-Sample Ultra 2.0 User Guide: Prepare samples and digest with Proteinase K steps 1.a. and b., page 3). 480 μL of the Proteinase K Mix was added to each well containing a swab (as the corresponding urine pellets had been done, in accordance with the MagMAX™ DNA Multi-Sample Ultra 2.0 User Guide: Prepare samples and digest with Proteinase K step 1.d., page 3). At the same time, the following deviations from the User Guide are performed: 480 uL of water was pipetted into an empty well (according to the batch plate map) to serve as a “no template control”; 480 uL of the Proteinase K Mix was also pipetted into an empty well to serve as an “extraction control” (according to a batch plate map). After that, the plate was sealed with clear adhesive film and shook at 900 rpm for 5 minutes, according to the MagMAX™ DNA Multi-Sample Ultra 2.0 User Guide: Prepare samples and digest with Proteinase K step 1.e. Then the plate was removed from the shaker and the sealed plate was incubated for 30 minutes (another deviation from the MagMAX™ DNA Multi-Sample Ultra 2.0 User Guide) at 65 degrees Celsius.

From this point, the protocol (the MagMAX™ DNA Multi-Sample Ultra 2.0 Kit User Guide: Perform DNA purification using KingFisher Flex, steps 1 through 4) can be followed with the following two modifications: Instead of the 96-well standard KingFisher Flex heatblock was used according to the step 1, the 96 deep-well KingFisher heat block was used; and instead of 50 uL of Elution Solution was pipetted into a 96-well standard plate according to the step 2's table, 50 uL of Elution Solution was pipetted into a 96 deep-well plate.

In some embodiments, in order to save time and resources, for example, in order to avoid extraction, instead of utilizing an extraction kit and/or instrumentation, a one-step lysis procedure can be performed to free cellular contents in urine pellets and in corresponding buccal cells—particularly nucleic acids, which can then be amplified and comparatively analyzed to accurately identify samples. In these embodiments, extraction kits and automated extraction instrumentation can be avoided altogether by using a lysis buffer to break open urine epithelial cells and buccal cells, as well as nuclei in each, thus releasing DNA. More specifically, lysis buffers comprised of enzymes and detergents would dissolve cellular proteins and free DNA, at which point free DNA would be separated from cellular debris that might inhibit amplification by means of precipitation. Cell lysis buffers can purify DNA in a single step, often times at room temperature and in as little as a few minutes. DNA loss can be minimized by utilizing whole cell lysate as a starting material for PCR amplification. The quality and consistency of analysis results of the DNA isolated via this single-step lysis isolation method (direct lysate) may be subpar in comparison to analysis results of the DNA isolated via the optimized extraction. However, this single-step lysis DNA isolation method could be a faster and cheaper option adaptable for workplace drug testing spaces.

After DNA extraction and purification or lysis, but before analysis, DNA can be amplified and genotyped. Any thermal cycler could be utilized to amplify DNA from urine and buccal cells. In a preferable embodiment, an Applied Biosystems by Thermo Fisher Scientific Veriti 96 Well Thermal Cycler for polymerase chain reaction (PCR) DNA amplification can be purchased and used. Based on the principle of enzymatic replication of the nucleic acids, PCR is a method widely used to rapidly make millions to billions of copies of a specific DNA samples, allowing scientists to take a very small sample of DNA and amplify it to a large enough amount that it can be studied in detail.

Subsequently, the DNA from the two sources of samples can be analyzed and compared, and based upon the analysis and comparison results, a match or mismatch between a donor identity of the urine drug testing sample and the confirmed donor identity of the reference sample can be determined and reported. The analysis and comparison can be performed with any methods for analyzing single nucleotide polymorphisms (SNPs), including but not limited to one or any combination of the following: mass spectrometry (e.g. LC-MS/MS, GC-MS/MS, MALDI-TOF-MS, etc.), DNA sequencing, molecular beacons, SNP microarrays, PCR-based methods, random amplified polymorphic detection (RAPD), restriction fragment length polymorphism (RFLPI), amplified fragment length polymorphism detection (AFLPD), allele specific oligonucleotide (ASO) probes, etc.

In some preferable embodiments, in order to analyze and compare the DNA from the two sources of the urine drug testing sample and corresponding buccal cell sample, the iPLEX Pro Sample ID Panel kit from Agena Bioscience, Inc. can be purchased and used, which was designed to screen DNA extracted from fresh tissue, frozen tissue, FFPE samples, cell lines, blood plasma, serum, buccal cells and hair follicles when used with the MassARRAY System by Agena Bioscience for amplification and analysis (MALDI-TOF MS technology—matrix assisted laser desorption ionization-time of flight mass spectrometry), almost exactly according to the iPLEX Pro Sample Integrity Panels User Guide from Agena Bioscience, hereby incorporated by reference in its entirety with the following exceptions. Instead of using the iPLEX Pro Sample ID Panel with the MassArray for tissue identification, the panel is used for urine sample identification in clinical toxicology and/or workplace drug testing spaces. The iPLEX Pro Sample Integrity Panels User Guide can be used to meet needs by adding Coriell BioBank samples as quality controls to each batch prior to amplification and analysis, as well as by naming urine DNA samples and corresponding buccal cell samples in such a way that report generation with MassArray analytical software (Typer software) assigns “unexpected” or “expected” and “match” or “mismatch” to samples purported to have come from the same human based on results from 44 SNPs (single-nucleotide polymorphisms), 3 gender markers and 5 copy number controls.

Relatedly, acquisition parameters can be set for ideal data generation (since the samples analyzed are different from what the panel was designed for) within SpectroAcquire software (MassArray operation software), including but not limited to setting the “Start Dispense Condition” to 550, as opposed to the recommended parameters for the Sample ID panel. Additionally, specific limits may be defined within Typer analytical software for assigning “match” or “mismatch” to samples.

The foregoing description and accompanying FIGURES illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. A method for verification of a donor identity of a urine sample, comprising:
preparing a urine sample and a reference sample, wherein a donor identity of the reference sample is confirmed to be a person providing the urine sample, and wherein the reference sample is a buccal cell sample, a urine sample, a whole blood sample, a tissue sample, a saliva sample and/or an oral rinse sample from the person;
extracting or releasing DNA from the urine sample and the reference sample simultaneously, wherein the DNA from a urine sample is gDNA from epithelial cells excreted into the urine;
amplifying and genotyping the DNA from both samples simultaneously, wherein the amplification is performed by polymerase chain reaction (PCR);
after the genotyping, analyzing and comparing the DNA from the urine sample to the DNA from the reference sample, wherein the analysis and comparison is performed through one or more methods of analyzing single nucleotide polymorphisms (SNPs) selected from the group consisting of mass spectrometry (MS), DNA sequencing, molecular beacons, SNP microarrays, PCR-based methods, random amplified polymorphic detection (RAPD), restriction fragment length polymorphism (RFLPI), amplified fragment length polymorphism detection (AFLPD), and allele specific oligonucleotide (ASO) probes; and
based upon the analysis and comparison results, determining, and reporting a match or mismatch between a donor identity of the urine sample and the donor identity of the reference sample.

2. The method according to claim 1, wherein the reference sample is a buccal cell swab.

3. The method according to claim 1, wherein the DNA from a urine drug testing sample is gDNA from epithelial cells excreted into the urine.

4. The method according to claim 1, wherein DNA extraction from the urine sample and the reference sample is performed by commercially available DNA extraction kits, with or without automated DNA extraction instruments.

5. The method according to claim 4, wherein the extracting the DNA from the urine sample and the reference sample is performed by commercially available DNA extraction kits intended for buccal cell DNA extraction with automated DNA extraction instruments.

6. The method according to claim 5, wherein DNA extraction from the urine sample and the reference sample is performed with the same kit on a same plate at the same time.

7. The method according to claim 6, wherein the extracting of the DNA from both samples on the same plate at the same time comprises: digesting the cells from both samples with Proteinase K mix, and performing DNA purification.

8. The method according to claim 1, wherein the releasing of DNA from the urine sample and the reference sample is to release the DNA through a one-step lysis procedure to free cellular contents in the samples instead of performing an extraction with a kit and instrumentation.

9. The method according to claim 8, wherein the one-step lysis procedure comprises using a lysis buffer to break open the cells from the urine sample and the reference sample, as well as nuclei in each of the cell types, thus releasing the DNA for amplification and subsequent analysis and comparison.

10. The method according to claim 9, wherein the lysis buffer is formed of enzymes and detergents.

11. The method according to claim 1, wherein the analysis and comparison of the DNA from the urine sample and the reference sample are performed by matrix assisted laser desorption ionization-time of flight mass spectrometry (MALDI-TOF MS).

12. The method according to claim 1, wherein the donor identity of the reference sample is confirmed through polymerase chain reaction (PCR) and mass spectrometry (MS).

13. A method for identification of a donor of a urine sample, comprising:
preparing a urine sample and a reference sample, wherein a donor identity of the reference sample is confirmed to be a person providing the urine sample, and wherein the reference sample is a buccal cell sample, a urine sample, a whole blood sample, a tissue sample, a saliva sample and/or an oral rinse sample from the person; and
extracting or releasing DNA from the urine sample and the reference sample;
wherein the extracting of the DNA from the urine sample and the reference sample comprises: digesting cells from both samples with Proteinase K mix, and performing DNA isolation for amplification and subsequent analysis and comparison;
or wherein the releasing of the DNA from cells in the urine sample and the reference sample comprises: performing a one-step lysis procedure using a lysis buffer to free cellular contents simultaneously in both of the urine and reference samples at room temperature, as well as nuclei in each cell types, thus releasing DNA for amplification and subsequent analysis and comparison; and
wherein sample identification is confirmed through amplification by polymerase chain reaction (PCR) and analysis and comparison by one or more methods selected from the group consisting of mass spectrometry (MS), DNA sequencing, molecular beacons, SNP microarrays, PCR-based methods, random amplified polymorphic detection (RAPD), restriction fragment length polymorphism (RFLPI), amplified fragment length polymorphism detection (AFLPD), or allele specific oligonucleotide (ASO) probes.

14. The method according to claim 13, wherein the reference sample is buccal cell swabs.

15. The method according to claim 13, wherein the lysis buffer is formed of enzymes and detergents.

16. The method according to claim 13, wherein the DNA from the urine sample is DNA from epithelial cells excreted into the urine, or DNA from white blood cells in urine.

17. The method according to claim 13, wherein the releasing of the DNA from the urine and the reference sample is performed without an extraction kit or an automated extraction instrument.

18. The method according to claim 13, wherein the releasing of the DNA from the urine and the reference sample is performed by using extraction kits with or without automated extraction instrumentation.
